(19) United States
(12) Patent Application Publication
(10) Pub. No.: US 2023/0280358 A1
(43) Pub. Date: Sep. 7, 2023

(54) GUIDING BLOOD PRODUCT ADMINISTRATION TO MITIGATE BLEEDING RISK

(71) Applicant: CHILDREN'S MEDICAL CENTER CORPORATION, Boston, MA (US)

(72) Inventors: Sitaram Emani, Newton, MA (US); Sirisha Emani, Newton, MA (US); Juan C. lbla, Medfield, MA (US); Meena Nathan, Watertown, MA (US); Vishnu Sankar Emani, Newton, MA (US)

(73) Assignee: CHILDREN'S MEDICAL CENTER CORPORATION, Boston, MA (US)

(21) Appl. No.: 18/014,901

(22) PCT Filed: Jul. 8, 2021
(86) PCT No.: PCT/US2021/040832

§ 371 Date: Jan. 6, 2023

Related U.S. Application Data
(60) Provisional application No. 63/050,668, filed on Jul. 10, 2020.

(51) Int. Cl. G01N 33/86

(52) U.S. Cl. CPC G01N 33/86; G01N 2800/52

(57) ABSTRACT

Techniques disclosed herein provide a method of informing blood product administration to a perioperative patient. Some patients may be at risk of perioperative bleeding following a surgery, such as a cardiac surgery. As discussed herein, coagulation status of a patient is monitored perioperatively and at least one value indicative of the coagulation status may be used to determine the patient’s risk of perioperative bleeding. Should the patient’s risk of perioperative bleeding be determined to be sufficiently high, such as when the value(s) are below one or more thresholds, techniques disclosed herein may also in some embodiments aid in titration of a blood product to be administered to the perioperative patient to adjust the patient’s risk of bleeding.

### BACKGROUND

Cardiac surgery is surgery performed on the heart or adjacent vessels. Cardiac surgeries may be broken down into three phases: the preoperative stage, the intraoperative stage, and the postoperative stage. During the preoperative stage, examinations are performed, medication is provided, and a patient is generally prepared for the surgical procedure to be performed. The intraoperative stage begins when the patient is received in the surgical area. During this phase, the patient is anesthetized and the surgical procedure is performed. The postoperative stage begins after the patient is transferred to the intensive care unit (ICU) following the surgical procedure. At the ICU, the patient is monitored and receives additional care.

Some cardiac procedures may include cardiopulmonary bypass (CPB). CPB is a technique in which a machine functions as the heart and lungs of the patient such that it circulates and oxygenates the blood of the patient.

For some cardiac surgeries, such as during a surgery including a CPB, hypothermia may intentionally be induced in the patient to prevent neurological damage. Additionally, an anticoagulant (blood thinner) such as heparin may be administered during some cardiac surgeries. Following such surgeries, the body temperature of the patient may be gradually brought back to baseline during a rewarming and/or a medication, such a protamine, may be administered to counteract the effects of an anticoagulant.

### SUMMARY

In one embodiment, there is provided an apparatus comprising at least one processor and at least one computer-readable storage medium having encoded thereon executable instructions that, when executed by the at least one processor, cause the at least one processor to carry out a method to inform perioperative blood product transfusion. The method comprises, in response to determining that at least one value indicative of a coagulation status of a perioperative patient is below a first threshold, determining a volume of platelets to transfuse to the perioperative patient based at least in part on the at least one value and outputting a recommendation to administer the volume of platelets to the perioperative patient.

In another embodiment, there is provided at least one computer-readable storage medium having encoded thereon executable instructions that, when executed by the at least one processor, cause the at least one processor to carry out a method to inform perioperative blood product transfusion. The method comprises, in response to determining that at least one value indicative of a coagulation status of a perioperative patient is below a first threshold, determining a volume of platelets to transfuse to the perioperative patient based at least in part on the at least one value and outputting a recommendation to administer the volume of platelets to the perioperative patient.

In a further embodiment, there is provided A method to inform perioperative blood product transfusion. The method comprises, in response to determining that at least one value indicative of a coagulation status of a perioperative patient is below a first threshold, determining a volume of platelets to transfuse to the perioperative patient based at least in part on the at least one value and outputting a recommendation to administer the volume of platelets to the perioperative patient.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

FIG. 1 shows an example of a system in which some techniques described herein may be performed.

FIG. 2 depicts a flowchart of an illustrative technique that may be performed by a bleeding risk evaluation facility according to some embodiments, to determine whether a patient may be at risk of bleeding and/or may benefit from administration of a blood product.

FIG. 3 depicts a flowchart of an illustrative technique that may be performed by a bleeding risk evaluation facility according to some embodiments, to determine a dosage of a blood product to be administered to a patient.

FIG. 4 shows percent of patients reaching the composite end-point in OR and ICU. A) Patients (%) reaching composite end-pint in the OR based on maximum amplitude by TEG comparison between rewarming and post-protamine. Patients (%) reaching composite end-pint in the ICU in patients who did or did not receive platelet transfusion with (B) MA<45 mm and (C) MA≥45 mm. *p<0.05 was considered statically significant.

FIG. 5 shows Bland-Altman plots for agreement of all TEG parameters comparing rewarming (33° C.) versus post-protamine (>35° C.). The Gray dotted lines represent mean bias values. Upper and lower dot lines represent 95% confidence intervals of upper and lower limits of agreement, respectively.

FIG. 6 shows Bland-Altman plots for agreement of all TEG parameters comparing TEG®5000 versus TEG®6S. Again, gray dotted lines represent mean bias values. Upper and lower dot lines represent 95% confidence intervals of upper and lower limits of agreement, respectively.

FIG. 7 shows linear regression (A) and Bland-Altman plots (B) comparing TEG-FLEV versus Clauss-Fibrinogen (mg/dl). Upper and lower dot lines represent 95% confidence intervals of upper and lower limits of agreement, respectively.

FIG. 8 shows linear regression models comparing A) percent increase in MA from before and after transfusion versus volume of platelet transfusion(ml/kg) (Pearson r:0.43; Y= 0.2559X + 6.637) and B) volume of platelet transfusion(ml/kg) versus difference in fibrinogen levels before and after platelet transfusion (Pearson r: 0.7; Y= 8.589X- 8.462).

FIG. 9 is a block diagram of an exemplary computing device with which some embodiments may operate.

### DETAILED DESCRIPTION

Techniques disclosed herein provide a method of informing blood product administration to a perioperative patient. Some patients may be at risk of perioperative bleeding following a surgery, such as a cardiac surgery. As discussed herein, coagulation status of a patient is monitored perioperatively and at least one value indicative of the coagulation status may be used to determine the patient’s risk of perioperative bleeding. Should the patient’s risk of perioperative bleeding be determined to be sufficiently high, such as when the value(s) are below one or more thresholds, techniques disclosed herein may also in some embodiments aid in titration of a blood product to be administered to the perioperative patient to adjust the patient’s risk of bleeding.

Some perioperative patients may be at risk of bleeding due to, for example, restricted coagulation as a result of anticoagulants administered to the patient during a surgery, including a cardiac surgery. To offset that risk, medications to counteract the anticoagulants may be administered. In some cases, blood products, such as platelets, may additionally be administered to the patient to further offset the risk of bleeding. However, if a patient’s blood has too high a concentration of platelets, the patient may be put at risk of thrombosis (e.g., formation of a blood clot). Since thrombosis also presents risks to the patient, it is undesirable to administer platelets unnecessarily or administer too great a dosage of platelets to the patient.

The inventors have recognized and appreciated that, conventionally, there was no mechanism to guide a clinician in titrating blood product administration to a perioperative patient at risk of bleeding, to reduce their risk of bleeding. For example, conventionally, there was no methodology for titrating administration of platelets to a patient. Instead, clinicians would need to guess at whether a patient might benefit from administration of a blood product like platelets, and administer a generic dose to a patient. This led to some patients not being administered a blood product when they may have benefited from one to reduce the risk of bleeding, and some patients being administered more blood product than needed. Both had negative consequences for patients, due to either continued risk of bleeding or increased risk of thrombosis. Despite the consequences, the problem has long persisted, because clinicians had no other option.

The inventors have recognized and appreciated that, however, that these difficulties could be mitigated through determining a patient’s risk of bleeding based on a patient’s perioperative coagulation status. More particularly, the inventors recognized and appreciated that if a patient’s perioperative coagulation status were measured, at least one value indicative of that coagulation status could then be used to determine the patient’s risk of bleeding. Based on the determined risk of bleeding, a determination could be made of whether blood product should be administered to the patient.

Moreover, the inventors recognized and appreciated that such value(s) may also be used to determine and output a recommended dosage of blood products to administer to the patient. For example, once one or more values indicative of a patient’s perioperative coagulation status have been determined, the value(s) may be used to determine a dosage of a blood product that, when administered to the patient, would change the patient’s coagulation status to a target status. This may include, for example, changing the value(s) to match target value(s) indicative of a coagulation status. The determination may be made, for example, using a trained model of how coagulation status is impacted by administration of dosages of blood products.

For example, in some embodiments, at least one value indicative of a perioperative coagulation status of a surgical patient could be compared to a first threshold and a second threshold greater than the first threshold. In some such embodiments, in instances wherein the value(s) are lower than the first threshold, the techniques disclosed herein may recommend administering a particular dosage of a blood product such that the patient’s coagulation status reaches a target status. For example, the dosage may be such that, when the value(s) for the patient are measured again, the value(s) have a desired level or are above a threshold (which may be the same or different threshold(s) than were used to determine the risk of bleeding). If, in some such embodiments, the value(s) indicative of the coagulation status are instead greater than the second threshold, the techniques disclosed herein may recommend not administering blood products. In some embodiments, in instances wherein the at least one value indicative of the coagulation status of a patient is between the first and second thresholds, the techniques disclosed herein may utilize additional data to arrive at a determination of whether or not to administer a dosage of blood product such that the value(s) increase to a target.

Such techniques may be used to determine a perioperative patient’s risk of bleeding following a surgery. Such a surgery may be, for example, a cardiac surgery, though embodiments are not limited in this respect. In addition, the surgical patient may be, for example, a pediatric patient. In some embodiments discussed below, coagulation status of the patient may be measured perioperatively, including post-operatively, and the determination may be made based on that measurement. In some such embodiments, the measurement may be taken during a rewarming of the patient, or following administration of a medication to counteract the effects of an anticoagulant that had been administered to the patient.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

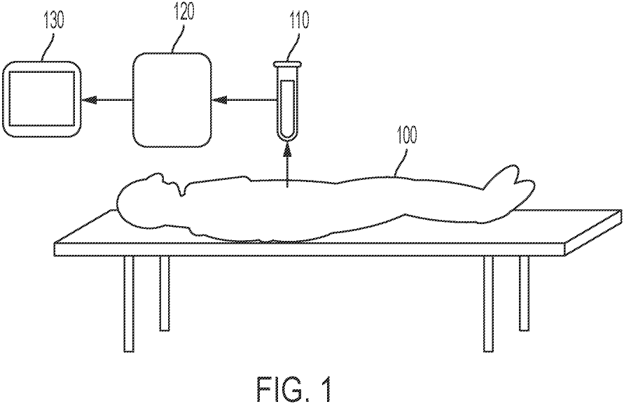

FIG. 1 shows a blood sample 110 drawn from perioperative surgical patient 100. The blood may have been drawn, for example, during or following a surgery, such as during a rewarming following the surgery or following administration of a medication to counteract the effects of an anticoagulant that had been previously administered to the patient 100. At least some of the blood sample 110 may be analyzed using device 120 to determine a coagulation status of the patient 100. While a human patient 100 is illustrated, some embodiments are not so limited. In the depicted embodiment, the device 120 is configured to execute a bleeding risk evaluation facility that is implemented as executable code, such as one or more pieces of software. The device 120 may perform one or more assays using the blood sample 110 to produce at least one value indicative of the coagulation status of patient 100 at the time the blood was drawn from the patient 100. Based on the value(s), the bleeding risk evaluation facility may determine and output a recommended dosage of blood products to administer to the patient 100. The recommendation may be output via an interface of a device 130. Any suitable output interface may be used, as embodiments are not limited in this respect. For example, in some embodiments, the output may be audible, visual, and/or haptic, and the output may be via a graphical, audible, and/or haptic interface, or other forms of output and other forms of user interface. When the output is via an interface of device 130 (and/or, as discussed below, an interface of the device 120), the output may be via one or more displays, lights, speakers, or other hardware interface components of the device.

Device 120 may be any suitable device configured to analyze a blood sample. For example, device 120 may be configured to perform one or more assays on blood, corresponding to one or more particular blood analyses. Accordingly, the device 120 may be configured to receive at least some of the blood sample 110 and to perform one or more analyses on that blood. It should be appreciated that embodiments are not limited to performing the analyses in any particular manner and that any form of analysis may be performed, including one or more assays. In some embodiments, the analysis/analyses may include exposing the blood to one or more chemical stimuli (e.g., reagents), one or more mechanical stimuli (e.g., rotation), or other stimuli. The device 120 may also be configured to receive at least some of the blood sample 110 in any suitable manner, as embodiments are not limited in this respect.

The analysis/analyses the device 120 may be configured to perform include analyses relating to coagulation status of a patient from which an analyzed blood sample was drawn, such as patient 100 of FIG. 1. In some embodiments, the analysis/analyses of coagulation status may include thromboelastography (TEG), and the device 120 may determine one or more values indicative of coagulation status based on a TEG analysis performed on at least a portion of the blood sample. For example, the device 120 may be configured to determine, as a value indicative of coagulation status, a maximum amplitude (MA) of the TEG for the blood sample 120. Any suitable technique for performing TEG may be used in these embodiments, as these embodiments are not limited to a particular technique for performing TEG.

The device 120 may be located proximate to the patient 100 or may be remote from the patient 100, as embodiments are not limited in this respect.

The device 120 may also be configured to execute a bleeding risk evaluation facility, which may operate in accordance with techniques described herein to determine whether a patient is at risk of bleeding and, if so, whether to administer a blood product. The facility may make such a determination, in some embodiments as described herein, based on one or more values indicative of coagulation status of the patient 100. For example, the facility may make the determination based (at least in part) on the MA of the TEG of the blood sample 110. The facility may also, in some embodiments, generate a recommendation of a dosage of a blood product to be administered to patient 100. Examples of operations of such a facility are described below. As discussed below, in some embodiments, the facility may recommend whether to administer platelets to a patient at risk of bleeding and, in some embodiments, may recommend a dosage of platelets to administer.

In the embodiment of FIG. 1, output from the facility executed by the device 120 (e.g., determinations of whether a patient is at risk of bleeding, and/or whether and how much of a blood product to administer) may be transmitted to and output via a separate device 130. Embodiments are not so limited; in some embodiments, the facility’s output may be provided via an interface of the device 120. In the illustrated embodiment of FIG. 1, the device 130 may be any suitable computing device, including a web server, a laptop or desktop personal computer, a mobile computing device (e.g., a smart phone, tablet, or wearable computing device), or a medical device. In embodiments in which the device 130 may be a medical device, the medical device may also titrate a blood product. Accordingly, in some embodiments, the device 130 may be configured to titrate a blood product to the patient 100. For example, the medical device may be configured to titrate platelets to the patient 100 and may be configured per the recommendations output by the bleeding risk analysis facility executed by the device 120. The configuration may, in some embodiments, be automatic and the device 130 may titrate a blood product per the output of the facility without additional intervention by a clinician or other user. In other embodiments, the device 130 may be configured based on the output of the facility but may not begin administering or titrating a blood product without additional authorization input from a clinician or other user.

While not illustrated in FIG. 1, devices 120, 130 may communicate via any suitable computer communication techniques. For example, devices 120, 130 may communicate via one or more personal-area, local-area, and/or wide-area, wireless and/or wired communication networks. Such network(s) may include an enterprise network, such as for a medical facility (e.g., a hospital) and/or may include the Internet. In some embodiments, a same entity may own and/or operate devices 120, 130, while in other embodiments different entities may own and/or operate devices 120, 130.

While the example of FIG. 1 includes different devices 120, 130, it should be appreciated that embodiments are not so limited. In some embodiments, the same device may perform the operations described herein as being performed by devices 120, 130. In some embodiments that include two devices, functionality may be distributed between devices 120, 130 in any suitable manner. For example, while examples described herein include the bleeding risk analysis facility executing on the device 120, in some embodiments the facility may execute on device 130. For example, in some embodiments the device 120 may be configured to perform one or more analyses on the blood sample 110 and output results of the analyses. Those results may then be input (manually or through computer-based communication) to a bleeding risk evaluation facility executing on device 130, and the results of an evaluation by the facility may be output to a user via an interface of the device 130. In addition, in some embodiments, there may be more devices. For example, in some embodiments a web server (not pictured in FIG. 1) may be configured to execute the bleeding risk evaluation facility and a user may operate device 130 to interact with the facility via the web and view on the device 130 output recommendations of the facility.

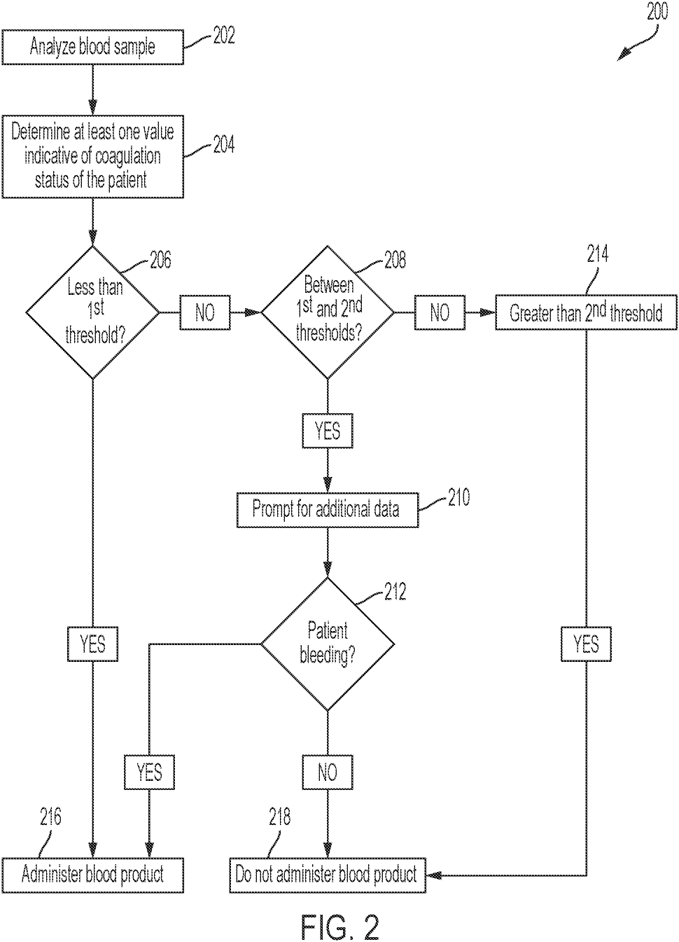

FIG. 2 depicts one possible method of operation for the aforementioned bleeding risk evaluation facility. Prior to the start of the process 200 of FIG. 2, a blood sample is obtained from a perioperative patient and is loaded into a device configured to perform an analysis on the blood sample. In some embodiments, including the embodiment of FIG. 2, the device that analyzes the blood sample may be configured to execute the bleeding risk evaluation facility as discussed above in connection with FIG. 1, though embodiments are not so limited.

The process 200 of FIG. 2 begins in block 202, in which the device analyzes at least some of the blood sample and, in block 204, produces at least one value indicative of the coagulation status of the patient.

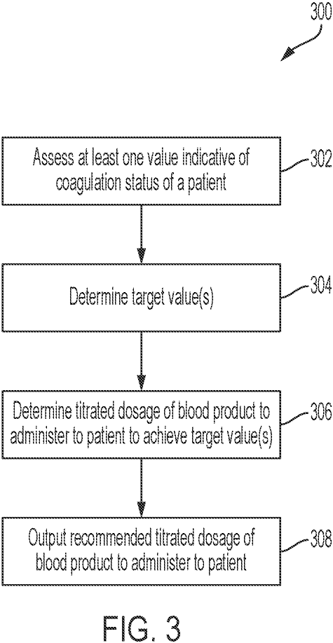

Next, in block 206, the bleeding risk evaluation facility compares the value(s) to at least one first threshold to determine whether the value(s) are less than or equal to the first threshold(s). If the facility determines the value(s) is below the first threshold 206, then in block 214 the facility recommends administering a blood product to the patient. As discussed above and in more detail below, the facility may determine a dosage of blood product to be administered, which in some embodiments may be done in accordance with techniques described in connection with FIG. 3 and other examples below.

If the facility determines that the value(s) are above the first threshold, the facility may determine in block 208 whether the value(s) is below the second threshold. Then in block 210, the facility may prompt for additional data. For example, the facility may prompt for input of whether the patient is bleeding. In some embodiments, as an alternative to or in addition to prompting for additional data, the facility may analyze previously-input data, such as additional data that was input when the blood sample was provided to the device for analysis in block 202. When additional data is input (previously and/or in response to a prompt), each part of the additional data may be analyzed according to what the additional data is. For example, if the additional data is whether the patient has been observed bleeding, then the additional data may be analyzed to determine whether the patient is bleeding or not. The facility performs such analysis of the additional data in block 212. As illustrated in FIG. 2 (in connection with the example where the additional data is whether the patient is bleeding), if the facility determines in block 212 that the patient is bleeding, then in block 216 the facility recommends administering blood product. If not, then in block 218 the facility outputs a recommendation not to administer a blood product.

While the bleed status of the patient is considered in the depicted methodology of FIG. 2, it should be appreciated that any appropriate patient condition, status, or metric may be considered in addition to or instead of the bleed status of the patient. For example, alternate methodologies may consider the weight, blood pressure, or another factor of the patient in addition to or instead of the bleed status of the patient.

If in block 208 the facility determines that the value(s) are not between the first and the second threshold, then in block 214 the facility determines that the value(s) are above the second threshold and outputs in block 218 a recommendation not to administer a blood product. The facility may recommend against administering a blood product in this case because the coagulation status of the patient may thus indicate that the patient is not at risk of bleeding. When the facility so determines that the patient is not at risk of bleeding, administration of a therapeutic blood product to counteract a risk of bleeding may not be necessary and, in some cases, may even be risky as it may put the patient at risk of thrombosis.

The example of FIG. 2 was discussed without a specific type of values indicative of coagulation status. In some embodiments, the value(s) indicative of the coagulation status of the patient may include at least one value based on a thromboelastography (TEG) measurement. For example, the value may be the maximum amplitude (MA) value of a TEG measurement. In some instances, the value(s) may be indicative of the patient’s coagulation status at a particular time relative to a surgery (e.g., at a particular milestone or phase of the surgery), by obtaining a blood sample and performing a blood analysis at the particular time or phase during or after the surgery. For example, in some embodiments, the time or phase may be a time following administration to the patient of a medication to counteract effects of an anticoagulant (e.g., protamine). As another example, the time or phase at which the blood sample may be drawn may be during rewarming of the patient following surgery.

In some embodiments in which the at least one value indicative of the coagulation status is the MA value of a TEG measurement, the first threshold may be that the value is between 40 mm and 50 mm, or below 50 mm. For example, the first threshold may be between 43 mm and 47 mm or below 47 mm, or between 44 mm and 46 m or below 46 mm, or equal to 45 mm. For example, in some embodiments the MA value of a TEG measurement may be evaluated to determine whether it is equal to or less than 45 mm. In other embodiments, the first threshold may be below 40 mm or greater than 50 mm.

Additionally, in some embodiments in which the at least one value indicative of the coagulation status is the MA value of a TEG measurement, the second threshold may be between 50 mm and 65 mm, or above 50 mm. For example, the second threshold may be between 53 mm and 57 mm or above 53 mm, or between 54 mm and 56 mm or above 54 mm, or equal to 55 mm or another value greater than the first threshold. For example, in some embodiments the MA value of a TEG measurement may be evaluated to determine whether it is equal to or greater than 55 mm.

While examples of specific thresholds have been given in connection with the example of the value indicative of a coagulation status being the MA of a TEG measurement, it should be appreciated that embodiments are not limited to operating with these particular thresholds and that others are possible.

FIG. 3 provides an example that may be used in connection with the methodology of FIG. 2. In the example of FIG. 2, the facility output a recommendation of whether to administer a blood product or not. With the technique illustrated in FIG. 3, the facility may in some embodiments output a recommendation of a dosage of blood product to administer.

The process 300 of FIG. 3 begins with the bleeding risk evaluation facility assessing in block 302 at least one value indicative of the coagulation status of a surgical patient. Should this initial value(s) assessment indicate that a blood product should be administered, as described above (e.g., the value is below a first threshold), the facility determines in block 304 one or more target value(s)for the value(s) indicative of a coagulation status of the patient that were assessed in block 302. The target value may be a value that, if achieved, would indicate that the patient’s coagulation status is within normal range and thus represent a reduced likelihood of postoperative bleeding. The target value may also be such that the patient’s coagulation status does not indicate a risk of thrombosis or other predisposition to clotting. The desired level may therefore reduce or minimize risk of an adverse outcome, such as bleeding or thrombosis.

The facility may determine the target value in block 304 in any suitable manner, including by obtaining the target value from storage. In some embodiments, a single target value may be used for all patients. In other embodiments, the target value may be selected based on one or more factors, such as patient-specific information such as demographics like age, gender, ethnic background, or others, or health factors such as weight, medical background (e.g., current or past medical conditions), or others. In some embodiments, the target value may be the same as the second threshold evaluated in block 302 and discussed above in connection with FIG. 2, though embodiments are not so limited.

Once the target value is determined 304, the facility determines in block 306 a dosage of blood product to administer to the patient that would cause the at least one initial value indicative of the patient’s coagulation status to increase to the target value(s). Examples of ways in which the dosage may be determined are discussed below. In some embodiments, a trained model may be used that accepts as input the current value(s) indicative of the patient’s coagulation status and outputs a dosage that is predicted to cause the value(s) to increase to the target value. Such a model may operate in a variety of suitable manner, examples of which are described below. For example, a trained regression analysis may be used in some embodiments.

Once the dosage is determined, in block 308 the facility outputs the recommended dosage of blood product.

In some embodiments, the target value(s) indicative of the coagulation status of a patient may be greater than the first threshold. Furthermore, in some instances, the target value(s) may be greater than or equal to the second threshold. For example, in embodiments in which the at least one value indicative of the coagulation status of a patient is the MA value of a TEG measurement, the target value may be between 51 mm and 67 mm, or greater than 67 mm. For example, the second threshold may between 53 mm and 63 mm, or greater than 63 mm. In some embodiments, the target value(s) may be 55 mm, or greater than 55 mm. In some embodiments, the target value is static in that the target value(s) is the same for all patients regardless of factors including age, weight, type of surgery performed, etc. In other embodiments, the target value(s) is variable in that different patients have different target value(s) based upon any number of suitable factors.

The recommended dosage of blood product may be selected such that the initial value of the at least one value indicative of the coagulation status of a patient, which may be indicative of the coagulation status of the patient during the perioperative phase of a surgery prior to protamine administration, is brought to the target value(s). In some instances, dosage may be chosen such that the initial value(s) increases to the target value(s). For example, in embodiments wherein the at least one value indicative of the coagulation status of a patient is the MA value of a TEG reading, and wherein the initial reading was below a first threshold such that the bleeding risk evaluation facility determined blood product transfusion was warranted and output an appropriate dosage of titrated blood product, the appropriate dosage may increase the initial MA value to a target MA value greater than the initial value.

While not illustrated in FIGS. 2-3, in some embodiments, the bleeding risk evaluation facility may utilize additional data in combination with the at least one value indicative of the coagulation status of a patient, to determine whether blood product should be administered to a patient and, if so, determine the specific dosage of blood product to recommend for administration. For example, the bleeding risk evaluation facility may additionally evaluate data such as the patient’s age, weight, or blood pressure, the type of surgery performed, any underlying health conditions of the patient, whether the patient has been observed bleeding, the type and/or dosage of anticoagulant administered, and/or other factor(s). Once data has been input, the facility may perform a regression or other analysis to determine an amount of blood product that, when administered to the patient, would result in the patient achieving a target value(s) for the at least one value indicative of the coagulation status of the patient.

In some instances, the bleeding risk evaluation facility may be configured to recommend the administration of a particular dosage of platelets based on the value of the at least one factor indicative of the coagulation status of the patient. In instances where the bleeding risk evaluation facility determines that a titrated dosage of platelets should be administered, the bleeding risk evaluation facility may recommend a volume of platelets to transfuse. The facility may additionally recommend a minimum volume of platelets to transfuse. In some instances, the volume of platelets to be transfused may be between 0.0 units and 80.0 units. In addition to or as an alternative to recommending a volume of platelets to transfuse, in some embodiments, the bleeding risk evaluation facility may additional output a volume of fibrinogen that may be transfused. In alternate embodiments, a dosage of a different blood product, medicine, or therapeutic may be recommended.

### Exemplary Embodiments

Discussed below is a research study performed in connection with exemplary embodiments of the technology discussed above. It should be appreciated that embodiments are not limited to operating in accordance with the examples described below in this section, as other embodiments are possible.

Some embodiments disclosed herein may in some instances be utilized to inform blood product administration for pediatric patients, although it should be appreciated the techniques disclosed herein may be applicable to all patients regardless of age.

Thromboelastography (TEG) may be used to predict bleeding in pediatric patients undergoing surgery, including for example, complex cardiac surgery. In some instances, one or more TEG values obtained from blood drawn from the patient during the rewarming phase of cardiopulmonary bypass (CPB) may be used to guide intraoperative blood product management.

In a retrospective study to study effectiveness of a particular illustrative embodiment, TEG values obtained during rewarming and after protamine administration were compared using TEG®5000 and TEG®6S. A composite endpoint of extended blood product transfusion or surgical re-exploration for bleeding was utilized as a surrogate for post-operative bleeding. Bleeding in the operating room (OR) was distinguished from bleeding in the intensive care unit (ICU).

The aforementioned study was performed in an operating room (OR) and cardiac intensive care unit (ICU) at a single institution. The subjects of the study were703 pediatric (≤18 yrs) patients undergoing complex cardiac surgery requiring CPB who underwent perioperative TEG testing. No interventions were performed in the study.

By multivariable analysis, longer CPB time and lower TEG maximal amplitude (MA) during rewarming were independently associated with risk of composite endpoint in the OR or ICU (p<0.05). Among patients with MA less than 45 mm during rewarming, those who received platelet transfusion in the OR were less likely to reach composite endpoint within the subsequent 24 hrs in the ICU compared to those who did not receive intraoperative platelet transfusion (10% vs. 32% respectively; p<0.01). Good correlation was observed between TEG parameters at rewarming vs. after protamine administration. Similarly, good correlation was observed between TEG®5000 vs. TEG®6S at various time points. The relationship between platelet transfusion volume (ml/kg) and percent change in MA between rewarming and post-transfusion time-points was determined using linear regression and a platelet transfusion calculator was generated.

The study showed that in pediatric cardiac surgery patients, lower MA during rewarming is associated with increased risk of perioperative bleeding. In patients with rewarming MA less than 45 mm, intraoperative platelet transfusion may reduce the risk of subsequent bleeding. Tailored platelet transfusion therapy based on rewarming TEG may reduce the risk of bleeding while minimizing unnecessary platelet transfusion.

Complex cardiac procedures requiring cardiopulmonary bypass (CPB) in the pediatric population may result in perioperative bleeding requiring blood product transfusions. As per the STS database, greater than 50% of patients undergoing complex cardiac surgery procedures utilize blood product transfusions peri-operatively Development of a guided perioperative blood product regimen may reduce unfavorable post-operative bleeding while minimizing blood product administration.

Commonly utilized diagnostic techniques to monitor coagulation status during cardiac surgical procedures include partial thromboplastin time (aPTT), activated clotting time (ACT), thromboelastography (TEG) and rotational thromboelastometry (ROTEM). Unlike aPTT and ACT, viscoelastic techniques (TEG and ROTEM) provide a multi-component analysis of coagulation, including coagulation factor function, platelet and fibrinogen function, as well as clot lysis. Limited data is available regarding utility of TEG in pediatric patients following cardiac operations. It has been previously reported that TEG maximal amplitude (MA) measured after administration of protamine is associated with peri-operative bleeding. Apart from the basic TEG parameters, the other crucial factor associated with unfavorable perioperative outcomes is fibrinogen levels. However, since blood product administration typically occurs in the first 30 minutes following protamine administration, sampling TEG at the post-protamine time point is not practical to guide intraoperative blood product transfusion as time to result is approximately 30-45 min.

In this study, the hypotheses was tested that TEG parameters measured during the rewarming phase of cardiopulmonary bypass: (1) demonstrate association with endpoints of perioperative bleeding; (2) are available by the time of protamine administration; and (3) correlate with post-protamine TEG parameters. Based on TEG results, a blood product management algorithm was developed, including a web-based platelet transfusion calculator to estimate platelet transfusion volume to be administered peri-operatively. TEG parameters measured by TEG®5000 and TEG®6S were compared in anticipation of using TEG®6S since the latter can be utilized at point-of-care in the OR during rewarming requiring lower volume and is less operator-dependent.

### Materials and Methods

### Study Design

After approval by the Institutional Review Board, consecutive pediatric (age ≤ 18 years) patients undergoing cardiac surgery with cardiopulmonary bypass (CPB) at Boston Children’s Hospital between August 2016 and December 2019 were retrospectively screened. Patients were included in this study if they were considered to be high risk for bleeding according to following inclusion criteria: neonatal operation (age ≤ 30 days), single ventricle physiology, reoperative sternotomy with greater than two previous operations, complex cardiac reconstruction with prolonged duration of CPB. Patients were excluded if they did not undergo perioperative laboratory testing for TEG in the operating room (OR) at rewarming and post-protamine time-points or if patients received blood product transfusion prior to TEG testing in the OR.

A total of 703 patients who met inclusion and exclusion criteria were divided into two separate cohorts. Cohort 1 (537 patients-training group) was used to develop a risk model for bleeding and the platelet transfusion calculator, while the Cohort 2 (166 patients-validation group) was used to validate the developed risk model and calculator. The validation cohort includes patients who were only given platelet transfusion in the OR and excludes all patients given other blood products in addition to platelet transfusion.

### Sample Collection and Testing

Whole blood samples were collected at several time points peri-operatively. The first samples were collected during rewarming on CPB at 33° C. The second samples were collected after protamine administration at greater than 35° C. The third samples were taken following blood product administration in the cardiac intensive care unit (ICU). TEG was performed on all three samples using a TEG®5000 device (Haemonetics Corp, Braintree, MA) in a CLIA certified laboratory. When available, discarded whole blood samples from TEG®5000 testing were utilized for thromboelastography on TEG®6S (Haemonetics Corp, Braintree, MA) device performed concurrently.

### Data Collection

Patient characteristics, blood product transfusions and clinical laboratory data including all TEG®5000 parameters along with Clauss-fibrinogen levels were retrospectively collected from the medical record. Where available the same TEG parameters along with functional fibrinogen (FLEV) were collected for TEG®6S.

A composite endpoint was established as a surrogate for perioperative bleeding that included transfusion requirements or need for surgical re-exploration for bleeding as described previously. The composite endpoint was further characterized as being reached intraoperatively (in the OR following protamine administration but prior to transfer to ICU) or within 24 hours (excluding intraoperative events, within 24 hours) of arrival to ICU.

### Blood Product Transfusions Calculator

Using Python libraries, least-squares regressions were performed in order to model the relationship between MA and peri-operative platelet transfusion. The Pearson correlation r test was implemented on the various prediction models, and best fit model was found to be a linear fit.

A linear regression model was generated based on percent difference in TEG maximum amplitude (MA) before and after platelet transfusion versus volume of platelet transfusion (ml/kg). Based upon previous studies, the target volume of platelets to transfuse was estimated based on reaching a target MA of 55 mm. For TEG®6S, a correction factor was included based on a linear regression model for MA developed between TEG®5000 versus TEG®6S. A linear regression equation estimating fibrinogen levels in platelet transfusion was generated based on percent difference in fibrinogen levels before and after platelet transfusion versus volume of platelet transfusion (ml/kg). TEG-FLEV was corrected to the Clauss-fibrinogen results using a linear regression equation.

The calculator was implemented into user-friendly website that yields a recommended platelet transfusion volume as well as estimated fibrinogen based upon user inputs, for example, TEG data and patient weight.

### Statistical Methods

Categorical data were described as number with frequency and continuous data as median with interquartile range (IQR). Association between patient variables including TEG parameters and composite bleeding endpoints was assessed by multivariable logistic regression. Bland-Altman plots were constructed by plotting average values of both measurements against difference between the measurements for all comparisons. Association between TEG parameters in OR and composite endpoint in ICU was determined by using Fisher exact test. Statistical analysis was performed using Python libraries (NumPy) and GraphPad Prism version 7.0 (San-Diego, CA). A p-value less than0.05 was considered statistically significant.

### Results

### Patient Characteristics and Outcomes

Patient characteristics for Cohort 1 (training cohort, N=575) and Cohort 2 (validation cohort, N=166) are presented in Table 1.

TABLE 1

Patient demographics and characteristics

| Patient Characteristic | Median (IQR) or N (%) | |
|---|---|---|
| | Training Cohort (N=537) | Validation Cohort (N=166) |
| Age at surgery (months) | 15 (3, 49) | 9 (3, 34) |
| Neonates | 79 (14%) | 18 (11%) |
| Weight (kg) | 9.2 (5, 15) | 7.8 (5, 13) |
| Body surface area (m²) | 0.4 (0.3, 0.6) | 0.4 (0.3, 0.6) |
| Male | 323 (60%) | 93 (56%) |
| CPB time, time (min) | 147 (103, 196) | 163 (123, 222) |
| Cross-Clamp time, time (min) | 99 (69, 132) | 109 (84, 158) |
| Composite endpoint reached in OR | 136 (25%) | N/A |
| Composite endpoint reached in ICU | 51 (10%) | 27 (16%) |

Median (IQR) or number (%) are represented in the table. IQR: Inter Quartile Range; CPB: Cardiopulmonary bypass; OR: Operating room; ICU: Intensive care unit

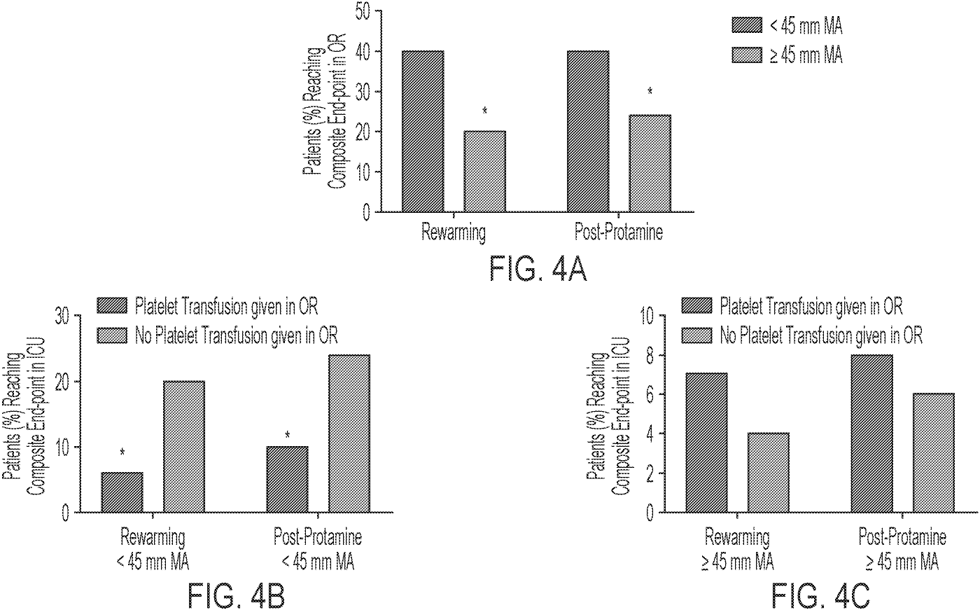

Among Cohort 1, the composite endpoint was reached in the OR in 136/537 (25.3%) and within 24 hours of ICU admission in 51/537 (9.5%) of patients (FIG. 4). For the validation cohort, composite endpoint was reached post-operatively (within 24 hrs of ICU admission) in 27/166 (16.2%).

### Association Between TEG Parameters and Bleeding

Within Cohort 1, by univariate analysis, CPB time, age at surgery, and TEG MA during rewarming were discovered to be significantly associated with risk of reaching composite bleeding endpoint in the OR and ICU (Tables 2 and 3). Multivariable logistic regression analysis determined that longer CPB time and lower MA were independent risk factors for bleeding (both OR and ICU). The median R-time, K-time, and angle were not significantly different between patients who reached and did not reach composite endpoint. However, the median MA in patients reaching composite endpoint was 47.6 mm (43.2, 51.8) compared to 50.3 mm (46.5, 53.3) in patients who did not reach the composite endpoint (P<0.001). Analysis of MA by TEG®5000 during rewarming demonstrated that a higher proportion of patients reached the composite endpoint intraoperatively when MA was less than 45 mm compared to MA greater than equal to 45 mm (45/119 [37.8%] vs. 91/418 [21.8%], P<0.001) (FIG. 4A). Iterative analysis with various rewarming MA values revealed that MA of 45 mm was a viable discriminator between bleeding and non-bleeding patients in the OR.

TABLE 2

Multivariable logistic regression model results for bleeding in the OR (N=537, Bleeding N=136)

| Models | Odds ratio | 95% CI | P-value |
|---|---|---|---|
| Age (months) | 1.0 | 1.0, 1.01 | 0.1 |
| Cardiopulmonary Bypass time (min) | 1.0 | 1.0, 1.01 | <0.01 |
| Maximum amplitude, mm | 0.94 | 0.91, 0.98 | <0.01 |

TABLE 3

Multivariable logistic regression model results for bleeding in the ICU (N=537, Bleeding N=51)

| Models | Odds ratio | 95% CI | P-value |
|---|---|---|---|
| Age (months) | 1.0 | 1.0, 1.01 | 0.1 |
| Cardiopulmonary Bypass time (min) | 0.99 | 0.99, 1.0 | 0.02 |
| Maximum amplitude, mm | 0.89 | 0.84, 0.95 | <0.01 |

### TEG MA Comparisons at Rewarming and Post-Protamine Time Points

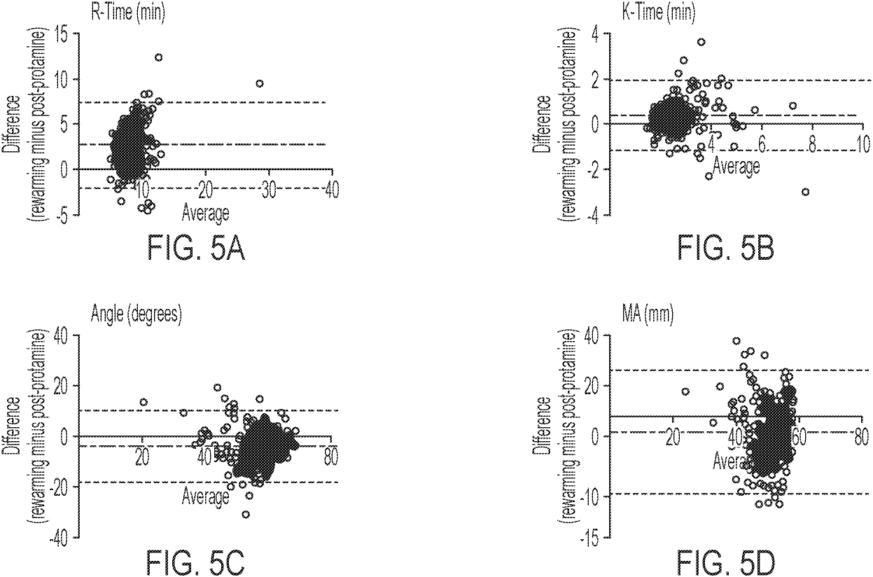

TEG MA values at rewarming and post protamine time points were compared. The R-time and K-time were longer and angle and MA were shorter during rewarming compared to post-protamine time point (paired t-test; p<0.05). There was good correlation between rewarming and post-protamine MA (Pearson r=0.76). Bland-Altman plots were constructed by plotting average values of both time points against difference (rewarming minus post protamine) between the values for both time points for all TEG parameters (FIGS. 5A-D). Table 4 shows the Bland-Altman parameters including bias values, limits of agreement (LOA), and percent coefficient of variation (%CV) for all TEG parameters. The %CV between rewarming versus post-protamine time-points was less than 15% for R-time and K-time, and less than 5% for angle and MA, suggesting good agreement between the two time points. As MA was determined as a viable TEG parameter associated with reaching the composite end point, the comparisons were therefore focused on MA.

TABLE 4

Bland-Altman parameters for agreement of limits: Rewarming versus Post-protamine and TEG®5000 versus TEG®6S

| TEG Parameter | Bias | SD of Bias | 95% LOA | %CV |
|---|---|---|---|---|
| R-Time (minutes) Rewarming vs. Post-protamine | 2.7 | 2.4 | -2.0 - 7.4 | 14.2 |
| K-Time (minutes) Rewarming vs. Post-protamine | 0.37 | 0.76 | -1.1 - 1.9 | 8.5 |
| Angle (degrees) Rewarming vs. Post-protamine | -3.9 | 7.2 | -18 - 10.3 | 4.3 |
| MA (mm) Rewarming vs. Post-protamine | -2.0 | 3.9 | -9.7 - 5.6 | 3.6 |
| R-Time (minutes) TEG®5000 versus TEG®6S | -0.2 | 1.8 | -3.8 - 3.4 | 2.1 |
| K-Time (minutes) TEG®5000 versus TEG®6S | -0.1 | 0.7 | -1.6 - 1.3 | 2.6 |
| Angle (degrees) TEG®5000 versus TEG®6S | -2.4 | 6.2 | -14.5 - 9.7 | 2.9 |
| MA (mm) TEG®5000 versus TEG®6S | 5.2 | 3.0 | -0.6 - 11 | 7.4 |

SD: Standard deviation; LOA: Limits of agreement; CV: Coefficient of variance

### Impact of Intraoperative Platelet Transfusion on Bleeding Endpoints in the ICU

The impact of intraoperative platelet transfusion on bleeding endpoints in the ICU was examined. Within Cohort 1, 319 of 537 (59.4%) patients received platelets intraoperatively prior to transfer to ICU. The impact of MA at rewarming and platelet transfusion in the OR upon composite endpoint of bleeding within 24 hrs of arrival to ICU was analyzed. Among 119 patients with MA less than 45 mm, 94 (79%) received platelet transfusion intraoperatively. The proportion of patients with rewarming MA less than 45 mm who subsequently reached composite bleeding endpoint within 24 hours in the ICU was lower in patients who received platelet transfusion in OR (7/94, 9.6%) compared to those who did not (8/25, 32%; p<0.01). (FIG. 4B). Among patients with MA greater than 45 mm at rewarming, 212 patients received intraoperative platelet transfusion. There was no significant difference in composite bleeding endpoint in the ICU when comparing patients who did or did not receive platelet transfusion in the OR (9.7% versus 7.3%, p=0.4) (FIG. 4C). Similar trends were observed when post-protamine MA was analyzed (FIG. 4).

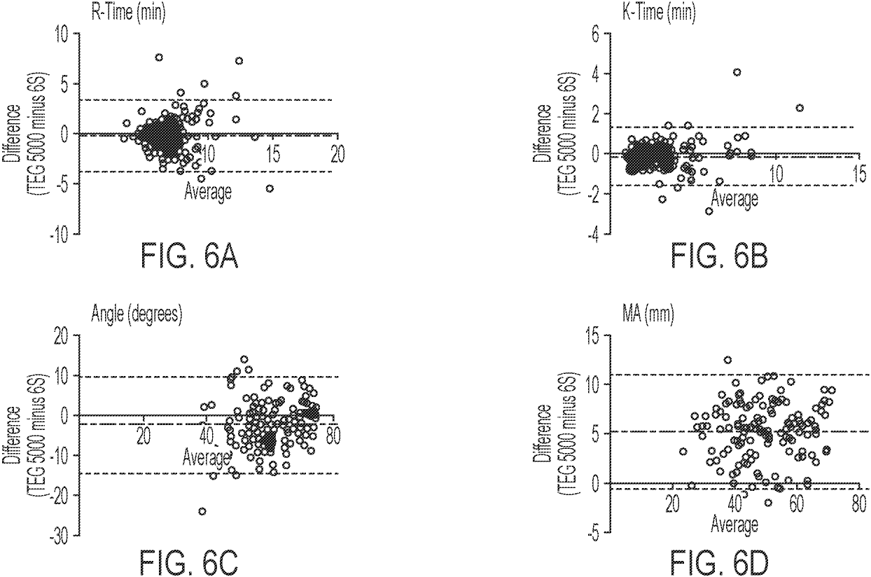

The TEG®5000 was compared to the TEG®6S. Bland-Altman parameters for limits of agreement between TEG®5000 and TEG®6S are shown in FIG. 6. Although there was a good correlation for MA values measured between the two devices (Pearson r=0.97), a consistent bias was detected by Bland-Altman plot, with lower values measured by TEG®6S compared to TEG®5000. The %CV was less than 10% between TEG®5000 versus TEG®6S for all TEG parameters, suggesting agreement between TEG®5000 and TEG®6S (Table 4). Average time to result for TEG®6S was 30 min, and TEG parameters were available in each patient at the time of protamine administration.

### Fibrinogen Measured by TEG®6S

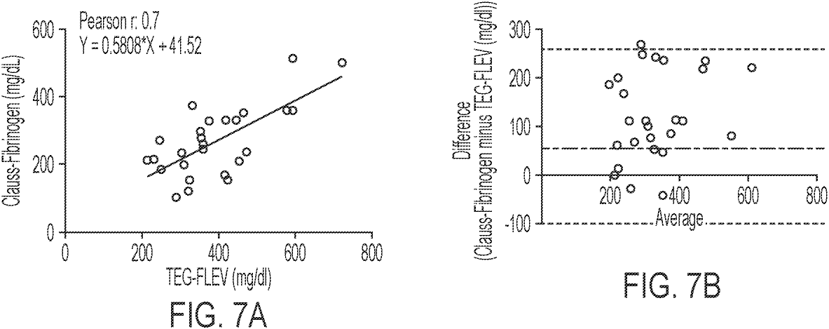

Fibrinogen measured by TEG®6S (TEG-FLEV) (median 360 mg/dl, (308,456)) were higher compared to the Clauss-fibrinogen (median 253 mg/dl, (195,337); p<0.001) and there was a moderate correlation between the two methods (Pearson r=0.7) (FIG. 7A). FIG. 4B shows Bland-Altman plots for TEG-FLEV values from both methods. The %CV between averages of Clauss-fibrinogen and TEG-FLEV was 27.4%. Once, TEG-FLEV was corrected to Clauss-fibrinogen using the linear regression equation, the %CV between averages of Clauss-fibrinogen and corrected TEG-FLEV was less than 0.01%.

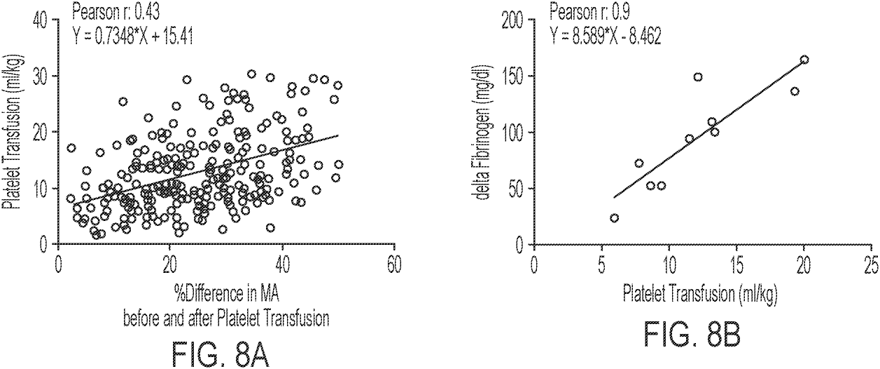

The effect of platelet transfusion on TEG parameters was examined. Based on values of MA measured before and after platelet transfusion in Cohort 1, a linear regression model was used to create a relationship between volume of platelet transfusion and change in MA (FIG. 8A). Using the linear regression equation, a platelet transfusion calculator was generated to help guide intraoperative platelet transfusion. The calculator was designed to estimate, based upon the rewarming MA, the volume of platelet transfusion to achieve a MA of 55 mm. The target MA of 55 mm was chosen based upon previous study that demonstrated this value as an inflection point in the relationship between probability of bleeding and MA. While a target value of 55 mm was chosen, it should be appreciated that any target value may be used as described previously.

The platelet transfusion calculator was validated in a separate cohort of 166 patients (Cohort 2) who received platelet transfusion following complex cardiac surgical procedures. Based upon the rewarming MA, ‘adequate’ platelet transfusion was defined as the platelet volume (ml/kg) that may achieve a post-transfusion MA of 55 mm. Adequate platelet transfusion in the OR was administered in 122 of 166 (73%) patients, whereas 44 of 166 (27%) patients were given smaller volume of platelet than suggested by the calculator (inadequate transfusion). Among patients with MA less than 45 mm who received inadequate platelet transfusion in the OR, 10 of 28 (35%) subsequently reached bleeding endpoint in the ICU, whereas only 6 of 43 (14%) patients who received adequate platelet transfusion in the OR subsequently reached bleeding endpoint in ICU (p<0.05).

In order to determine the impact of platelet transfusion upon plasma fibrinogen levels, TEG-FLEV was assessed in a subset of patients undergoing TEG testing prior to, and following, platelet transfusion. A linear regression model demonstrated an increase in TEG-FLEV following platelet transfusion, with an increase the fibrinogen levels of 8.5 mg/dl for every 1ml/kg platelet transfusion (FIG. 8B).

### Discussion

This retrospective study of pediatric patients undergoing high-risk cardiac surgery demonstrates an association between TEG parameters measured during rewarming phase of CPB at 33° C. and surrogate endpoints of bleeding within the early postoperative period (intraoperatively and within first 24 hrs of arrival to ICU). Also, patients with MA less than 45 mm at rewarming in the OR who did not receive platelet transfusion demonstrated a higher rate of bleeding in ICU compared to those who did receive platelet transfusion. Conversely, there was no significant risk of bleeding in the ICU in patients with normal MA, for example, MA greater than equal to 45 mm, in the OR who did or did not receive platelet transfusion. Based upon the findings of this study, an algorithm was created for intraoperative platelet transfusion has been developed that employs a platelet transfusion calculator.

Association between post-protamine MA and bleeding endpoints has been previously discussed; however, clinical application of post-protamine TEG has been limited by the delay in time to result. Many decisions regarding blood product administration occur within 30 min after protamine. A significant advantage of TEG measurement during rewarming is availability of the result at the time of protamine administration to guide blood product management. Similar to the previously published association between post-protamine MA and bleeding endpoints, MA during rewarming was the only laboratory parameter associated with perioperative bleeding. Although MA values were significantly different between rewarming and post-protamine, the difference was consistent among all samples, resulting in good correlation. Heparin samples are neutralized during TEG sample processing so that difference in TEG parameters observed between the two time points are most likely temperature effect.

Given the association between rewarming TEG and bleeding endpoints, the utility of rewarming MA to guide platelet transfusion was examined. A previous study demonstrated that MA of 55 mm was an inflection point beyond which the risk of postoperative bleeding plateaued. Excess administration of platelets and cryoprecipitate has been associated with higher risk of perioperative thrombosis. Therefore, a calculator was developed to provide an approximate volume of platelet transfusion to increase MA to this target value of 55 mm. Inadequate platelet transfusion in the operating room was associated with bleeding within 24 hours of arrival to the ICU, even if the patient was not bleeding in the operating room. Conversely, platelet transfusion was not associated with benefit if the MA was greater than 45 mm. In the validation cohort, the model confirmed that under-dosing based on the estimated calculation with techniques described herein leads to higher risk for bleeding in the ICU compared to adequate platelet transfusion dosing.

Accordingly, described herein are examples of an algorithm to guide platelet transfusion, developed based in part upon the results of this study. Patients may undergo TEG testing, including when they have been determined pre-operatively to be high risk for bleeding based upon inclusion criteria provided in this study. During rewarming phase of CPB, MA will be obtained to guide platelet transfusion. Following protamine administration, if bleeding is encountered in a patient with MA less than 55 mm, platelet transfusion can be guided according to the calculator. Regardless of bleeding status, if MA is less than45 mm, platelet transfusion can be administered according to calculator following protamine administration. In contrast, platelet transfusion may not be indicated for patient with MA greater than or equal to 45 mm in the absence of clinical bleeding.

To facilitate intraoperative workflow in use of rewarming TEG for clinical use, the correlation between TEG®6S and TEG®5000 was examined. Advantages of TEG®6S for pediatric applications include ease of use, low sample volume (340µl), ability to measure TEG-FLEV, and ease of maintenance. The automated sample processing performed by this device reduces operator variability and may prevent technical errors. Good correlation was observed between the two TEG devices at all time points, although a consistent 5 mm bias with TEG®6S MA values lower than TEG®5000 values was observed. Although there was correlation between TEG®6S-FLEV and Clauss-fibrinogen, a consistent bias was observed with higher values measured by TEG®6S-FLEV compared to Clauss-fibrinogen. Upon correcting for this difference between the methods using linear regression, there was improved agreement between the two methods (variance reduced from 27% to less than 0.01%).

The definition of bleeding in the study was based upon retrospective review of records and thus may be subject to inaccuracy. Although the sample size for developing the platelet transfusion calculator was adequate, there were limitations to the sample size for Clauss-fibrinogen versus TEG-FLEV comparisons. Since this study was performed in a specific pediatric population and from a single center, further studies may be needed to confirm that the results may be generalizable to other populations.

The inventors have therefore recognized and appreciated that low MA measured at rewarming is associated with increased risk for bleeding in pediatric patients undergoing high-risk cardiac surgery. Prophylactic platelet transfusions in patients with MA greater than or equal to 45 mm at rewarming may not be useful. TEG®6S would be a good alternative point-of-care method that is comparable to TEG®5000 for easy translation of correlation between MA and peri-operative bleeding outcomes.

Referring back to the figures, FIG. 1 shows the percent of patients reaching the composite end-point in OR and ICU. Patients (%) reaching composite end-pint in the OR based on maximum amplitude by TEG comparison between rewarming and post-protamine. Patients (%) reaching composite end-pint in the ICU in patients who did or did not receive platelet transfusion with (B) MA<45 mm and (C) MA≥45 mm. *p<0.05 was considered statically significant.

### Exemplary Computer-Implemented Embodiments

Techniques operating according to the principles described herein may be implemented in any suitable manner. Included in the discussion above are a series of flow charts showing the steps and acts of various processes that provide a method of informing blood product administration to a perioperative patient. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.. In other implementations, the functional facilities may be adapted to interact with other functional facilities in such a way as form an operating system, including the Windows® operating system, available from the Microsoft® Corporation of Redmond, Washington. In other words, in some implementations, the functional facilities may be implemented alternatively as a portion of or outside of an operating system. [pick relevant examples]

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or nonpersistent solid-state memory (e.g., Flash memory, Magnetic RAM, etc.), or any other suitable storage media. Such a computer-readable medium may be implemented in any suitable manner, including as computer-readable storage media 806 of FIG. 8 described below (i.e., as a portion of a computing device 800) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

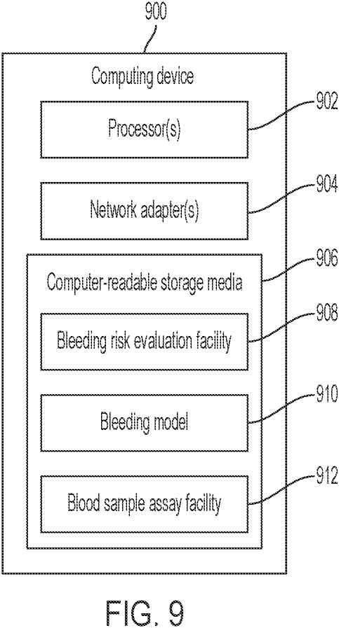

In some, but not all, implementations in which the techniques may be embodied as computer-executable instructions, these instructions may be executed on one or more suitable computing device(s) operating in any suitable computer system, including the exemplary computer system of FIG. 9, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 9 illustrates one exemplary implementation of a computing device in the form of a computing device 900 that may be used in a system implementing techniques described herein, although others are possible. It should be appreciated that FIG. 9 is intended neither to be a depiction of necessary components for a computing device to operate as an apparatus for recommending whether and how to administer a dosage of blood product to a patient in accordance with the principles described herein, nor a comprehensive depiction.

Computing device 900 may comprise at least one processor902, a network adapter904, and computer-readable storage media906. Computing device 900 may be, for example, a desktop or laptop personal computer, a personal digital assistant (PDA), a smart mobile phone, a server, a wireless access point or other networking element, or any other suitable computing device. Network adapter 904 may be any suitable hardware and/or software to enable the computing device 900 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 906 may be adapted to store data to be processed and/or instructions to be executed by the processor 902. The processor 902 enables processing of data and execution of instructions. The data and instructions may be stored on the computer-readable storage media 906.

The data and instructions stored on the computer-readable storage media 906 may comprise computer-executable instructions implementing techniques which operate according to the principles described herein. In the example of FIG. 1, the computer-readable storage media stores computer-executable instructions implementing various facilities and storing various information as described above. For example, the media 906 may store a bleeding risk evaluation facility 908 implementing one or a combination of the techniques described above. As another example, the media 906 may store a bleeding model 910, which may have been trained as described above and which may be used by the facility 908 in generating recommendations to output. The media 906 may also include a blood sample assay facility that, when executed, operates apparatus 900 to perform one or more assays or other analyses on a blood sample, including a TEG analysis using hardware components of or controllable by the device 900.

While not illustrated in FIG. 9, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

The indefinite articles “a” and “an,” as used herein in the specification and in the claims, unless clearly indicated to the contrary, should be understood to mean “at least one.”

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. An apparatus comprising:
at least one processor; and
at least one computer-readable storage medium having encoded thereon executable instructions that, when executed by the at least one processor, cause the at least one processor to carry out a method to inform perioperative blood product transfusion, the method comprising:
in response to determining that at least one value indicative of a coagulation status of a perioperative patient is below a first threshold,
determining a volume of platelets to transfuse to the perioperative patient based at least in part on the at least one value; and
outputting a recommendation to administer the volume of platelets to the perioperative patient.

2. The apparatus of claim 1, wherein:
the at least one value indicative of coagulation status of the perioperative patient comprises at least one value based on at least one thromboelastogram (TEG) measurement for the perioperative patient; and
determining the volume of platelets to transfuse to the perioperative patient based on the at least one value indicative of the coagulation status of the perioperative patient comprises determining the volume of platelets to transfuse based at least in part on the at least one value based on at least one TEG measurement.

3. The apparatus of claim 2, wherein:
the at least one value based on the at least one TEG measurement and indicative of the coagulation status of the perioperative patient comprises a maximum amplitude (MA) of a TEG measurement for the perioperative patient; and
determining that the at least one value indicative of the coagulation status of the perioperative patient is below the first threshold comprises determining that the MA of the TEG measurement for the perioperative patient is below the first threshold.

4. The apparatus of claim 3, wherein the first threshold is 45 mm.

5. The apparatus of claim 2, wherein the at least one value is indicative of the coagulation status of the perioperative patient is prior to protamine administration.

6. The apparatus of claim 5, wherein the method further comprises: performing at least one TEG measurement on a blood sample obtained from the perioperative patient prior to protamine administration, to generate the at least one value indicative of the coagulation status of the perioperative patient.

7. The apparatus of claim 1, wherein the method further comprises:
in response to determining that at least one value indicative of the coagulation status of the perioperative patient is between the first threshold and a second threshold higher than the first threshold,
in response to determining that the perioperative patient is experiencing bleeding, determining a volume of platelets to transfuse to the perioperative patient based at least in part on the at least one value.

8. The apparatus of claim 7, wherein the method further comprises:
in response to determining that at least one value indicative of the coagulation status of the perioperative patient is above the second threshold, outputting a recommendation that no platelets be transfused to the perioperative patient.

9. The apparatus of claim 8, wherein the second threshold is 55 mm.

10. The apparatus of claim 1, wherein the perioperative patient is a pediatric perioperative patient.

11. The apparatus of claim 1, wherein the method further comprises:
in response to determining the at least one value indicative of the coagulation status of the perioperative patient is below the first threshold, determining a volume of fibrinogen to transfuse to the perioperative patient based at least in part on the at least one value.

12. The apparatus of claim 1, wherein determining the volume of platelets to administer comprises performing a regression analysis to determine an amount of platelets that, when administered to the perioperative patient having the coagulation status indicated by the at least one value, would result in the perioperative patient having a target coagulation status.

13. The apparatus of claim 12, wherein:
the at least one value indicative of coagulation status of the perioperative patient comprises at least one value based on at least one thromboelastogram (TEG) measurement for the perioperative patient; and
determining the amount of platelets that, when administered to the perioperative patient would result in the perioperative patient having the target coagulation status comprises determining an amount of platelets that, when administered to the perioperative patient, would result in the perioperative patient having at least one second value, based on the TEG measurement, above a second threshold.

14. The apparatus of claim 13, wherein:
the at least one value based on the at least one TEG measurement and indicative of the coagulation status of the perioperative patient comprises a maximum amplitude (MA) of the TEG measurement for the perioperative patient;
the at least one second value comprises an MA of the TEG measurement; and the second threshold is 55 mm.

15. At least one computer-readable storage medium having encoded thereon executable instructions that, when executed by the at least one processor, cause the at least one processor to carry out a method to inform perioperative blood product transfusion, the method comprising:
in response to determining that at least one value indicative of a coagulation status of a perioperative patient is below a first threshold,
determining a volume of platelets to transfuse to the perioperative patient based at least in part on the at least one value; and
outputting a recommendation to administer the volume of platelets to the perioperative patient.

16. The at least one computer-readable storage medium of claim 15, wherein:
the at least one value indicative of coagulation status of the perioperative patient comprises at least one value based on at least one thromboelastogram (TEG) measurement for the perioperative patient; and
determining the volume of platelets to transfuse to the perioperative patient based on the at least one value indicative of the coagulation status of the perioperative patient comprises determining the volume of platelets to transfuse based at least in part on the at least one value based on at least one TEG measurement.

17. The at least one computer-readable storage medium of claim 16, wherein:
the at least one value based on the at least one TEG measurement and indicative of the coagulation status of the perioperative patient comprises a maximum amplitude (MA) of a TEG measurement for the perioperative patient; and
determining that the at least one value indicative of the coagulation status of the perioperative patient is below the first threshold comprises determining that the MA of the TEG measurement for the perioperative patient is below the first threshold.

18. (canceled)

19. The at least one computer-readable storage medium of claim 16, wherein the at least one value is indicative of the coagulation status of the perioperative patient is prior to protamine administration.

20-26. (canceled)

27. A method to inform perioperative blood product transfusion, the method comprising:
in response to determining that at least one value indicative of a coagulation status of a perioperative patient is below a first threshold,
determining a volume of platelets to transfuse to the perioperative patient based at least in part on the at least one value; and
outputting a recommendation to administer the volume of platelets to the perioperative patient.

28-36. (canceled)

37. The method of claim 27, wherein the method further comprises:
in response to determining the at least one value indicative of the coagulation status of the perioperative patient is below the first threshold, determining a volume of fibrinogen to transfuse to the perioperative patient based at least in part on the at least one value.

38. (canceled)
